(19) United States
(12) Patent Application Publication
(10) Pub. No.: US 2020/0022397 A1
(43) Pub. Date: Jan. 23, 2020

(54) Nutrient Support for Hormonal Forms of Contraception

(71) Applicant: Sarah Morgan, Aurora, CO (US)

(72) Inventor: Sarah Morgan, Aurora, CO (US)

(21) Appl. No.: 16/517,641

(22) Filed: Jul. 21, 2019

Related U.S. Application Data
(60) Provisional application No. 62/701,763, filed on Jul. 21, 2018.

(51) Int. Cl. A23L 33/15; A23L 33/175; A23L 33/16; A61K 45/06

(52) U.S. Cl. CPC A23L 33/15; A61K 45/06; A23L 33/16; A23L 33/175

(57) ABSTRACT

The present invention relates to a combination nutrient supplement directed to the remuneration of nutritional deficiencies and biochemical imbalances, directly or indirectly, related to the use of exogenous hormones. The unique nutritional supplement that is the present invention is directed toward supporting the unique deficiencies of women taking exogenous hormones (e.g. hormonal contraceptives and hormone replacement) which supplant an identified subset of depleted and essential vitamins and minerals deficient in those women taking exogenous hormones which are vital to providing for a woman's health, wellness and overall homeostatic state.

### CROSS-REFERENCE TO RELATED APPLICATIONS

U.S. Provisional Application No. 62/701,763, filed Jul. 21, 2018

### FEDERALLY SPONSORED RESEARCH OR DEVELOPMENT

Not Applicable

### INCORPORATION BY REFERENCE OF MATERIAL SUBMITTED ON A COMPACT DISK

Not Applicable

### FIELD OF THE INVENTION

The present invention relates generally to a combination nutrient supplement for nutritional deficiencies and biochemical imbalances directly related to the use of hormonal contraceptives. Specifically, the nutritional supplement that is the present invention is directed toward supporting the unique dietary deficiencies of women taking oral contraceptives—including vitamins, minerals, antioxidants and mitochondrial functional supporters and microbiome balance facilitators—which replace an identified subset of essential vitamins and minerals, deficient in those women taking hormonal contraceptives, vital to providing for a woman's health, wellness and overall homeostatic state. It is a goal of this invention to minimize resulting undesired effects such as low libido, mood dysregulation, fatigue, breakthrough bleeding, headaches, weight gain, appetite changes, GI distress, sleep disruption, blood dyscrasias, hormone irregularities, nitric oxide imbalances, dysbiosis and skin disorders (e.g. acne and dermatitis) that may be associated with premenstrual syndrome (PMS) or may occur at any time during the use of hormonal birth control.

### BACKGROUND

The development and mass production of hormone-based contraceptives has created an unparalleled paradigm shift in the way in which woman, and men, have chosen to procreate. Indeed, rarely has any one single chemical entity, or combination of entities, so radically affected such a dramatic societal change—providing both a means to simultaneously curb the population growth, maintain social and economic order and allow women a reversable means of contraception, sexual independence and liberation from constant and, theretofore, relatively unpredictable childbirth.

According to the United Nations, over 60 percent of partnered, reproductive-age women worldwide, representing about 740 million couples worldwide, practice some form of contraception—oral contraceptives accounting for about ⅙ of modern contraceptive practices which includes sterilization, prophylactics, IUDs, implants and injectable contraceptives. In fact, oral contraceptives, primarily containing both estrogen and progestin, are one of the most commonly utilized medications with over 100 million users worldwide.¹ Therefore, it is little wonder that reference to “the pill” requires no further context as it is used successfully and ubiquitously the world over.

Since the inception of the idea of hormonally regulated birth control, evidenced in 1921 by Ludwig Haberlandt's groundbreaking finding that ovaries transplanted from a pregnant rabbits and guinea pigs would foster a temporary infertile (sterile) period in a non-pregnant rabbit, hormonal contraceptive use has been highly controversial. The first US oral contraceptive was not introduced until 1957 (with the then approved indications of menstrual disorders and irregularities) as Enovid®, containing 10 mg of norethynodrel and 150 μg of mestranol. Enovid®, though, did not receive its approved indication of oral contraception by the US Food and Drug Administration (FDA) until Jun. 23, 1960.^{2,3} Although FDA approved, the stigma for OC use for married couples would not be relieved until 1965^{3,4} and not for unmarried woman not until 1972⁵.

Although, used for other purposes (e.g. irregular menses, menstrual cramping, amenorrhea, acne, Premenstrual Syndrome (PMS), endometriosis, Primary Ovarian Insufficiency (POI) and Polycystic Ovary Syndrome (PCOS)), hormonal contraceptives are nonetheless directed overwhelmingly toward the specific goal of avoiding pregnancy through the induction of infertility. Concisely, combination hormonal contraceptives act by preventing ovulation where hormone levels of LH and FSH are suppressed, mid-cycle surge of LH is absent, endogenous steroid levels are diminished, and ovulation does not occur.⁶

Manifestly, hormonal contraceptives are singular therapeutic entities, residing in their own class, as they (1) exhibit extremely high rate of effectiveness, (2) produce non-remedial results, (3) exist in many formulations and doses, and are (4) taken by predominantly, young, healthy women. Indeed, among those woman ages 15-44 who have reported ever having intercourse and ever using contraceptives, 25.4% have used an injectable formulation (i.e. Depo-Provera®), 10.6% reported using a contraceptive patch, and almost 80% (79.3%) have, at some point, used a contraceptive pill.^{7,8} Further, according to data from the 2011-2013 National Survey of Family Growth (NSFG) on contraceptive use in the month of the interview, approximately 62% of that same age range (ages 15 to 44) of the total population (61.7% or 37.6 million of 60.9 million women in the US) were currently taking some form of contraceptive—the “pill” accounting for 16 percent of this population and contraceptive rings (Nuvaring®) or patches (Ortho-Evra®) accounting for 4.4 percent of this population.⁸

Currently underway, inventor would argue, is the second phase of the hormonal contraceptive revolution where the remediation of certain vitamin and mineral deficiencies, through nutraceuticals and nutrient supplementation, is available to support the unique nutritional needs of women currently taking hormone-based birth control. This large segment of the female population has long endured anemias and dyscrasias directly or indirectly resulting from the depletion of or inability to produce and retain sufficient quantities of essential nutrients to maintain equipoise that can be seen to exacerbate underlying deficiencies which may develop gradually over months or even years. Indeed, supplementation of vitamins, minerals and other nutritional compounds and substances, that are absolutely essential to reducing or ameliorating hormonal contraceptive sequalae, achieve their goals through proper redox balancing in the mitochondria, regulation of hormonal metabolism, antioxidant activity within the cell, hormone detoxification, maintenance of proper hormone levels, nutrient absorption support and achieving and maintaining microbiome homeostasis manifestly warranted in the above population. To this end the present formulation and method of use is directed to the lessening of untoward effects that are inextricably linked to continued exogenous hormone use. It is the health, welfare and maintenance of this discrete population that marks an initiated step toward unencumbered use of O.C.'s (hormonal contraception), as well as hormone replacement, without unwanted side effects or with fewer side effects, that will create and maintain a woman's true reproductive independence.

Thus there is a significant and well recognized, and yet unmet, need in the art for a nutrient supplementation in the form of a nutraceutical formulation that provides for a restitutive composition and method for supplanting vital nutrients to those women choosing to take hormonal contraceptives as their primary means of birth control, and for other related restorative purposes, in an effort to remediate deficiencies that may be caused by oral contraceptives as well as other over the counter and prescribed pharmaceutical compounds wherein certain exogenous compounds can influence food intake and nutrient digestion, absorption, distribution, metabolism and excretion.⁹ The present invention satisfies this long-standing need in the art to address the diminution of deficient essential nutrients.

### BREIF SUMMARY OF THE INVENTION

The present disclosure provides a novel, scientifically derived and research-based means of restoring to women nutrients lost as a direct result of oral contraceptive (hormonal contraceptive) use and hormone replacement modalities and the untoward effects of deficiencies these losses create. Directly, these dietary insufficiencies carry with them a broad range of sequalae that effect a vast array of a woman's health ranging from sex drive, mood and energy to digestion, skin and hormone balance. It is therefore the purpose of the present invention to obviate the choice between taking reversible contraception (and hormone replacement) and avoiding the side-effects most commonly associated with exogenous hormones.

Specifically, the present formulation seeks to replenish lost nutrients, as identified by the World Health Organization, and targeted by inventor, of B₁ (Thiamine), B₂ (Riboflavin), B₃ (Niacin), B₅ (Pantothenic acid), B₆ (Pyridoxine), B7 (Biotin), B₉ (Folic Acid in the form of Methylfolate), B₁₂ (Methylcobalamin) and Vitamins C, & E as well as minerals Magnesium, Selenium and Zinc.⁸ Additionally, inventors have added organic broccoli sprout powder as a source of sulforaphane—an Nrf2 upregulator—to bolster the body's natural defense system, warding against cell damage and oxidative stress while acting to boost antioxidants and anti-inflammatory compounds, improving liver function, aiding in anti-cancer properties, and enhancing brain function. Too, inventor has included piperine (Bioperine) for enhanced nutrient absorption and bioavailability. Further, inventor has provided for the inclusion of calcium-d-glucarate to support the liver's ability to clear exogenous and endogenous hormones out of the body via excretion thereby lessening or avoiding the injurious effects of excess circulating hormones. In sum, the present formulation seeks to ameliorate and/or obviate the intractable effects of exogenous estrogens and progestins on the body by facilitating proper mitochondrial function and support, providing requisite nutrient transport, absorption, metabolism and excretion while working as a cellular antioxidant and hormonal detoxification mediator all while assisting in maintaining proper microbiome and biochemical balance within the body.^{10,11,12,13}

### DETAILED DESCRIPTION OF THE INVENTION

While advantages of the present invention will be readily apparent to those having skill in the art based on the appended description, there are described certain embodiments, formulations and strengths constituting the present invention and examples for illustrative purposes. And, although the following detailed description contains may specific references to ingredients and dosages, one having skill in the art will certainly appreciate that modifications, alterations and variations are within the scope of the present invention. Accordingly, the following embodiments of the invention are set forth without any loss of generality to, and without imposing limitations upon, the claimed invention. While preferred embodiments are described in connection with the description herein, there is no intent to limit the scope to the embodiments disclosed herein. On the contrary, the intent is to cover all equivalents.

The present invention provides a combination nutrient supplement directed toward abating and remediation of nutritional deficiencies and biochemical imbalances that satisfies the insufficiencies that oral, transdermal, IUD and injectable hormonal contraceptives create in women taking these forms of reversible hormonal birth control and other forms of exogenous birth control.

Accordingly, the nutraceutical combination that is the present formulation is designed to address, through prevention and treatment, the depletion of essential nutrients and the resulting biochemical imbalances that produce dysfunctional metabolic processes within the female body which may adversely affect mood, skin, hair, energy level, libido, gastrointestinal function related directly to hormone production, metabolism regulation and excretion and/or lack thereof.

Estrogen, as well as other estrogens (e.g. progestin), metabolism, both endogenous and exogenous, undergoes phase I (hydroxylation) and phase II (methylation) wherein the proper types and amounts of nutrients can positively or negatively influence each of several steps in the processing of estrogen. Namely, B2, B3, B6, B9, B12, and glutathione influencing the closely related and interdependent relationship between hydroxylation and methylation (where endogenous estradiol (E2), estrone (E1) and estriol (E3) are hydroxylated to 2-hydroxyestrone and methylated to 2-methoxyestrone and exogenous estrogens (e.g., 17α-ethinylestradiol—EE2) are hydroxylated to 4-hydroxyestrone and methylated to 4-methoxyestrone for use in the tissues), magnesium binds to COMT (Catechol O-methyltransferase) in an equilibrium sequence prior to the addition of SAM required in methylation and vitamins E (tocopherols), A (carotenes), C (ascorbic acid), beta-carotene (which converts to Vit. A), Zinc and selenium combine for reduced oxidation and free radicalization due to more polar, and water-soluble reactive oxygen intermediate metabolites existing between phase I and phase II thwarting detrimental effects on secondary tissues. Specifically, deficiencies in certain B Vitamins, B1 and B2 in particular, has been shown to decrease the ability of the liver to inactivate estrone and alpha-estradiol.¹⁹

In addition to the immediate effects of nutrient deficiencies, woman have been noted to exhibit increased signs of oxidative stress while on hormonal birth control thereby inviting elevated cardiovascular risk and resultant higher incidences of venous thromboembolism (including deep vein thrombosis (DVT), pulmonary embolism (PE), and cerebral thrombosis (CVT)) where higher estrogen doses, pregnancy, obesity, advanced age (over 40 years) and smoking may as well serve as contributory factors. This may be at least partially explained through lipid peroxidation and increases in catalase and glutathione peroxidase activities (i.e. endogenous antioxidant defenses) resulting in increased oxidative stress through increases in free radical activity marked by damage to fatty tissues, DNA, RNA, mitochondrial DNA and proteins. Increasing the body's antioxidant capabilities, though, can be seen to reverse the deleterious effects of oxidative stress that may be manifested in one, or a combination of, may varied and variable signs including headaches, fatigue, decreased neurotransmitter activity, gastrointestinal dysfunction, decreased liver function and resultant hormone metabolism insufficiencies and, above mentioned, obstinate cardiovascular health issues. Yet, supplanting the body with known antioxidants can retard or altogether obviate these deleterious effects whereby naturally occurring glutathione, and those cofactors supporting the production of glutathione (e.g. selenium, B2 (Riboflavin), Vitamin C and Vitamin E), operate to suppress or reverse recalcitrant effects of oral contraceptives.

Finally, hormonal contraceptives have not only been shown to deplete certain essential nutrients but also increase the levels of others, namely K2 (menaquinone), Vitamin A, Iron and Copper as well as alter the balance and ratio of calcium to magnesium. Untoward effects of excess K2 may include an increased blood plasma volume, which increases the thickness of your blood, thereby elevating blood pressure, CV disease risk and digestive distress. Hypervitaminosis A can lead to vision changes, skin changes and bone pain. Surplus iron can cause abdominal distress, aberrant blood clotting, menstrual cycle irregularities and may adversely affect heart, pancreas and liver functions. An overabundance of copper leads to fatigue, lack of appetite and abdominal pain and muscle stiffness. Advantageously, the current formulation and carefully selected strengths of formulation constituents serves to lessen or obviate these very symptoms. Of particular importance, though, is the relationship of calcium to magnesium where they may work together for muscle contraction and heartbeat or competitively and antagonistically where magnesium and calcium compete for the same binding site. This tenuous relationship may result in magnesium deficiency, precipitate excess calcium in the myocardium, increase the potential for clot formation, induce oxidative stress (with reduced participation of magnesium as an antioxidant with increases in calcium relative to magnesium) as well as lessen other advantageous effects of magnesium (e.g. proper muscle contractions, proper GI function, improved PMS symptoms, increased energy, strengthening of bones, improved heart health, lower blood pressure, proper blood sugar balance, improved stress response, ameliorated headaches/migraines, and better, more restful sleep).

The formulation itself consists of (1) B Vitamins (B1 (Thiamine), B2 (Riboflavin), B3 (Niacin), B5 (Pantothenic acid) B6 (Pyridoxine), B7 (Biotin), B9 (Folic Acid in the form of methyltetrahydrofolate), B12 (Methylcobalamin) (2) antioxidant Vitamins C & E (3) essential minerals, Magnesium, Selenium and Zinc in combination with (4) phytochemical sulforaphane and (5) alkaloid piperine and (6) calcium-d-glucarate as discussed below:

### B-Vitamins

While varying greatly in terms of structure and function, B vitamins are a group of water soluble vitamin that derive their designation in the “vitamin B complex” largely due to their isolation from the same source, namely liver and yeast.¹³ Each B Vitamin acts as either a cofactor/coenzyme (e.g. B₁, B₆, B₇ and B₁₂) or precursor to cofactors/coenzymes (e.g. B₂, B₃, B₅ and B₉).

The human gut microbiota and exogenous sources supply the human host with essential B-vitamins, including thiamin (B1), riboflavin (B2), niacin (B3), pantothenate (B5) and pyridoxine (B7), folate (B9) and cobalamin (B12), addressed herein. The human host relies largely on microbiota-derived B-vitamins from gut flora, as well as those obtained from dietary sources, as human cells alone do not produce sufficient quantities of B-vitamins to support normal functioning.¹³ Yet, any disruption in the intake of essential B Vitamins can have cascading detrimental effects on equipoise. Therefore, with a system already heavily reliant upon exogenous sources of Vitamin B sources, any endogenous dyscrasias or downward pressure on the presence of exogenous B Vitamins in the body threatens to have a great effect on an already tenuous reliance upon outside sources of B Vitamins.

### Vitamin B₁ (Thiamine)

Vitamin B1 is a primary example of an essential “helper” nutrient that cannot be synthesized by humans and must be derived through exogenous sources, or via gut flora, for use everywhere and in all tissues in the body for various and varied metabolic processes (including the mitochondrial pathways used to generate energy via the Krebs cycle and the Pentose Phosphate Pathway).¹⁴ By way of example, one liver cell can harbor as many as 2000 mitochondria, or cell power house, per hepatocyte—each hepatocyte, or liver cell, responsible for the creation of required body energy.¹⁵ Expressly, low levels of B1 in the digestive tract can lead to more lactate in the gut with symptoms of nausea, vomiting and abdominal pain which can, in some part, seek to explain the nausea routinely associated with OCs (hormonal contraceptives). Equally, the nervous system is dependent upon healthy B1 levels.^{16, 19}

Taking exogenous hormones (in the form of oral contraceptives) increases the need for vitamin B1 and/or depletes the overall B1 that the body is able to utilize.²¹ Moreover, Vitamin B1 is also imperative to making neurotransmitters (e.g. serotonin) for mood regulation, stabilization and proper functioning.^{16,17,20} Conversely, thiamine levels can affect the metabolism of hormonal contraceptives whereby thiamin deficiencies result in an increase metabolism rate in the area of three times as compared to non-thiamine deficient liver enzymes.^{18,19,21,24}

Symptoms of deficiency: lactate build up in GI tract associated with nausea and pain, irritability, depression, sleep disruption, muscle pain, fatigue, vision changes, memory dysfunction, irregular heart function and lack of alertness (in the case of beriberi resulting from the buildup of pyruvic acid) and nervous system, muscular system and visual and mental declines (in the case of Wernicke-Korsakoff syndrome)
Benefits: proper nervous system health, digestive function, efficient energy production, mood regulation, heart health and memory support and maintenance.

### Vitamin B2 (Riboflavin)

Riboflavin, like thiamine, is deficient in those women using oral contraceptivess.¹⁸ As with Vitamin B1, Vitamin B2 also evidences a neuroprotective function¹⁸ The bioavailable form our bodies prefer is R-5-P (riboflavin-5-phosphate) and riboflavin generally is used to make an important “helper” cofactors, FAD and FMN that serve as electron carriers and helps to regulate redox reactions and other metabolic pathways in the body. Low levels of riboflavin can as well impact vitamin B3 (niacin), B6, B9 (folate) and iron and tryptophan status. Women with MTHFR variants have higher needs for riboflavin as it is an important co-factor to this enzyme.

Riboflavin deficiency has been shown to increase oxidative stress (inflammation) in body and certain neurodegenerative disorders. As advanced by the Linus Pauling Institute, “[D]ecreased intracellular levels of flavocoenzymes could cause mitochondrial dysfunction, increase oxidative stress, and interfere with nitric oxide release and thus blood vessel dilation” which could contribute to headaches and migraines.²¹ Riboflavin also participates in the production of glutathione to regulate inflammatory control, immune balance and detoxification of endogenous and exogenous toxins via phase 1 and 2 detoxification in the liver.

Symptoms of deficiency: weakness, swollen tongue and throat, blurred vision, headache, itchy eyes, sensitivity to light, inflamed gums, skin irritation (dermatitis), increased blood pressure, anemia and fatigue.
Benefits: Increases in mitochondrial derived energy (cofactor to MTHFR), balanced nitric oxide levels, improve headaches and migraines²⁵, reduced inflammation in the body, support of eye, hair and skin health, decreases blood pressure and neuroprotective effects²²

### Vitamin B3 (Niacin)

Vitamin B3 is a helper in over 400 different enzyme reactions in the form of NAD and is crucial for DNA repair, mitochondrial function and genomic stability. Riboflavin deficiency can impact niacin status in the body. Niacin is needed to make important neurotransmitters like serotonin.

Symptoms of deficiency: fatigue, depression, headache, indigestion, canker sores, skin irritations
Benefits: mitochondrial health and adequate cellular energy production, healthy cholesterol levels, skin and hair health, enhanced mood, healthy digestion, weight management and proper gene expression

### Vitamin B5 (Pantothenic Acid)

Vitamin B5 is essential to formulate CoA for fat, carbohydrate and protein synthesis and metabolism and hormone generation and is also used to generate energy from the fat in our foods. Further B5 is integral to the synthesis of Co-enzyme A (CoA) which is vital to energy derivation for pyruvate entering the TCA cycle (tricarboxylic acid cycle) also titled the citric acid or Krebs cycle.

Symptoms of deficiency: irritability, fatigue, insomnia, depression, hair loss, stomach pain, and nausea and vomiting.
Benefits: assists body in coping with stress (make hormones), boosts immune system, helps liver function, aids in healthy hair and skin, weight management and proper gene expression.

### Pyridoxine—P-5-P (Also Known as Vitamin B6)

The bioavailable form of B6 that our bodies prefer is P-5-P (pyridoxal-5-phosphate).

Vitamin B6 is a helper for over 100 enzymes used in protein metabolism, nervous system health (e.g. neurotransmitter synthesis), hemoglobin synthesis and function (transport of oxygen), glucose metabolism, lipid metabolism, DNA production and gene expression, hormone regulation, histamine synthesis, mood-regulation, immune balancing and inflammatory controlling amino-acid tryptophan metabolism. It is a key nutrient which helps to keep homocysteine balanced (with the aid of zinc, B12 and folate) which contributes to vascular health and positive outcomes in pregnancy. Vitamin B6 must be synthesized in the plant kingdom by microbes or in other species that do not require Vit B6 and is metabolized in the liver as an essential dietary component. Absorption occurs almost exclusively in the GI tract. Additionally, B6 is a cofactor for reactions including transamination, decarboxylation, racemization and elimination and is responsible for the production of serotonin, melatonin, GABA, norepinephrine and dopamine.

MOA: Derangement of tryptophan metabolism occurs within 1 month of initiation of OC use. OCs also may cause a deficiency of pyridoxal phosphate, a coenzyme needed for the tryptophan-nicotinic acid pathway (J Nurse Midwifery. 1984 November-December; 29(6):386-90) The observed depression in plasma pyridoxal 5′-phosphate concentrations in OC users may reflect decreased body reserves of the vitamin, which could put women who discontinue OCs and become pregnant at risk for vitamin B6 inadequacy during pregnancy. (Nutr Rev. 2011 October; 69(10):572-83. doi: 10.1111/j.1753-4887.2011.00419.x) While the role that pyridoxine plays in cessation of nausea is incompletely understood, possible MOAs include prevention/treatment of vitamin B6 deficiency, intrinsic antinausea properties, and/or synergy with the antinausea properties of antihistamines (e.g. doxylamine) (Can J Anaesth. 2005 January; 52(1):55-61. Reeve et.al). Adding to this is the direct nausea inducing characteristics of estrogen and progesterone, alone and in concert, likely through a combination of direct effect, relaxation of the muscles in the stomach and intestines (through nitic oxide production), slow-wave gastric rhythm disruption and various metabolic and endocrinological factors related to hCG production, (Walsh Am J Physiol. 1996 March; 270(3 Pt 1):G506-14, Lee, Gastroenterol Clin North Am. 2011 June; 40(2): 309-vii. doi: 10.1016/j.gtc.2011.03.009)

In terms of mood, Vitamin B6 is a primary cofactor responsible for the conversion of tryptophan to 5-HTP (together with magnesium, calcium , iron and folate) which is then converted to serotonin (with the continued facilitation of B6 with Vitamin C, zinc and magnesium). This tryptophan-serotonin pathway thus benefits from B6 introduction at each essential step, wherein the final neurotransmitter product, serotonin, is responsible for sleep, sustenance, satiety, addiction, sexuality, CNS coordination, GI secretions, motility and tone and, most important to the present invention, emotions, stress and mood.

Symptoms of deficiency: worsening of PMS, sideroblastic anemia, impaired tryptophan-niacin conversion, impaired glucose tolerance, dermatitis, glossitis, anxiety, depression, fatigue, somnolence, neuropathy and muscle pain.
Benefits of supplementation: decreased nausea and vomiting, improved mood, more efficient processing of hormones, improved PMS symptoms (e.g. nausea), ameliorating anemia, reduction in inflammation, and maintenance of healthy blood vessels via regulation of homocysteine levels

### Biotin (Also Known as Vitamin B7)

Biotin is a key nutrient that helps our bodies use fats, carbohydrates and amino acids (protein) as energy via carboxylases along with regulation of gene expression. Adequate levels of biotin may help burn food as fuel and aid with issues of weight gain and weight control.

Symptoms of deficiency: fatigue, hair loss, dermatitis, depression, and neurological sequalae (tingling and numbness extremities, depression and lethargy).
Benefits: healthy cell growth, proper metabolism, glucose management, weight control, healthy skin, hair and nails and abatement of neurological symptoms.

### Vitamin B9(Folate/Folacin)

The bioavailable form our bodies prefer is methylfolate (5-MTHF) constituting 90% of what's in our bloodstream at any given time. B9, as noted in other B Vitamins, cannot be synthesized and levels are dependent on exogenous sources. Used to make healthy DNA, RNA and amino acids and is responsible for appropriate energy production (ATP) and red blood cell formation along with regulation of mood via the production of stimulating neurotransmitters—dopamine, epinephrine, norepinephrine as well as mood enhancing neurotransmitter serotonin, and the sleep facilitating neurotransmitter melatonin. Folate is vital for energy production in our muscles (creatine) as well as liver/gallbladder health via phosphatidylcholine production and regulation of inflammation in the body. Importantly, it has also been noted that hormonal contraceptives impair folate metabolism, as evidenced by folate deficiency in serum and an increase in urinary formiminoglutamic acid secretion. It is generally agreed that folate, which plays a critical role in fetal development, can become deficient in late pregnancy and in women who become pregnant shortly after discontinuing long term OC use (J Nurse Midwifery. 1984 November-December; 29(6):386-90)

### Methyltetrahydrafolate

Of note, methyltetrahydrafolate (5-methyltetrahydrafolate), as used in the formulation herein, has certain advantages over folic acid and folinic acid in that methyltetrahydrafolate reduces interactions with drugs that inhibit dihydrofolate reductase, prevents the potential negative effects of unconverted folic acid in the peripheral circulation, decreases the incidence of metabolic defects caused by methylenetetrahydrofolate reductase polymorphism and reduces the proclivity of other forms of folic acid to mask the hematological symptoms of vitamin B12 deficiency. It is therefore recommended that methyltetrahydrafolate be used in this formulation. Symptoms of deficiency: frequent infections from low immune system function, poor digestion, fatigue, changes in mood (e.g. depression), slow wound healing, anemia, and pale skin.

Benefits: proper DNA and RNA synthesis and repair, cell and tissue growth and repair, properly functioning immune system, lessening of inflammation, increasing detoxification, improved mood, sleep, improved brain function and supported heart health.

### Vitamin B12 (Cyanocobalamin)

B12, bioavailable as methylcobalamin, is an important partner nutrient to folate (folic acid) and it is used in the methylation cycle for healthy DNA production. Involved in the metabolism of every cell in the body, Vitamin B12 is also used to form the coating around nerves called the myelin sheath and, as such, is important for the normal functioning of the nervous system. Too, B12 is vital for the production of red blood cells in the bone marrow. Of importance to the present invention, gastric acid is required to process B12 and decreases in gastric acid levels leading to a lower bioavailability of B12 via consumed protein is observed in those patients taking H2-receptor antagonists and proton-pump inhibitors (prescribed in response to increased GI distress) wherein certain deficiencies (e.g. B1 insufficiencies and resultant lactic acid production) and direct effects (inherent hormone propensity to cause stomach upset) can equally contribute to reduced utilization of absorbed B12.

Symptoms of deficiency: weakness, fatigue, memory problems, shortness of breath, pale skin, indigestion, nerve problems, anxiety, depression.
Benefits: increased energy levels, memory, elevated mood, improved neurological pathways, healthy digestion, heart health via homocysteine balance, resilience to stress, improved genetic expression, and improved oxygen transport via red blood cells.

### Vitamins

### Vitamin C

L-ascorbic acid being the bioavailable form, Vitamin C is an important antioxidant in the body used to fight free radical damage and to boost levels of the body's main antioxidant, glutathione. Further, Vitamin C is an immune system modulator and booster. Vitamin C is a crucial nutrient for the production of carnitine which modulates inflammation in the mitochondria and helps to burn fat as energy (maintaining more stable blood sugars). Vitamin C is also needed to produce the neurotransmitters, dopamine, norepinephrine and epinephrine which provide increased energy and motivation. Vitamin C is also important for collagen production for tendons, ligaments and skin integrity.

Symptoms of deficiency: fatigue, weakness, weakened immune system, bloody nose, swollen joints, slowed healing, bleeding gums, iron deficiency anemia, mood disruption (depression), loss of collagen and poor skin appearance.

Benefits: boosts immune system, enhances mood and energy, minimizes inflammation, detoxification (due to high antioxidant activity), skin appearance, increases absorption of iron, burns fat as energy via production of carnitine.

### Vitamin E

Vitamin E is a fat-soluble antioxidant that protects cells, tissues and organs from free radical damage along with aiding in recycling glutathione. Vitamin E is especially important for protecting the skin, lungs and cell membranes. Vitamin E has also been shown to have a positive impact on immune system function. Vitamin E also improves sex drive by increasing testosterone.

Symptoms of deficiency: indigestion, lackluster hair, dry skin, inflammation, poor immune system function, weakness, vision problems, low libido and weakness
Benefits: improves skin appearance (e.g. dry skin), fights inflammation, provides detoxification from environmental exposures, minimizes PMS symptoms, fosters hormone balance, promotes shiny hair, provides improved skin appearance, enhances immune balance, lung health and libido

### Essential Elements

### Magnesium

Every unit of ATP (energy) production in your body requires magnesium. Poor magnesium intake equates to poor energy return. Magnesium is needed for approximately 300 biochemical reactions that maintain muscle and nerve function, potentiate energy production, prevent muscle cramps, supports heart rhythm, lowers oxidative stress, strengthens bones and regulate blood sugar levels.²¹

Crucial for energy production via ATP synthesis, bone and teeth structure, muscle contraction, magnesium provides various supportive functions including normal heart rhythm, nerve impulses, and electrolyte signaling at cell membrane. Magnesium is crucial for bone health (60% magnesium found in skeletal structure), heart health (27% magnesium found in muscles) healthy blood pressure, improving body's resilience to stress, wound healing, mood regulation and sleep.

Calcium regulation in the body is also dependent upon magnesium wherein is certain instances calcium works synergistically with magnesium and, at other times, calcium and magnesium work competitively and often at the same sites within the body. Unambiguously, levels of magnesium and levels of calcium are inextricably linked and ratios of one element to the other are dependent on the levels of each individually and the singular relationship calcium shares with magnesium where ratios falling outside of normally accepted ranges manifests itself in a series of secondary and tertiary, cascading and consequential metabolic events. Calcium as a mineral is a vital component of bones and teeth. The heart, nerves, and blood-clotting systems also require calcium to work properly. Calcium is also used for premenstrual syndrome (PMS), leg cramps in pregnancy, high blood pressure in pregnancy (pre-eclampsia), and reducing the risk of colon and rectal cancers. Expressly tied to the present invention, increased daily calcium, through proper maintenance or in relation to magnesium, appears to significantly reduce mood swings, bloating, food cravings, and pain. Also, increasing the amount of calcium in one's diet, or relationally through increases perceived circulating levels, appears to proportionately prevent or mitigate the signs and symptoms of PMS.

Specific to methylation of catechol estrogens to methoxy estrogens, via COMT (Catechol-O-methyltransferase), magnesium operates as an essential cofactor and, equally, as an antioxidant lowering the potential for DNA damage. Further COMT serves the additional function of breakdown of catecholamines (e.g. dopamine, norepinephrine and epinephrine) which serve as neurotransmitters and neuromodulators involved in the regulation of emotions and mood.

Symptoms of deficiency: muscle twitches and muscle cramping, fatigue, anxiety, depression, headaches, constipation, irregular heartbeat, hormone imbalance, insomnia, electrolyte imbalance, and low bone density.
Benefits: proper muscle contractions—decreased muscle spasms, proper digestion—relief of constipation, relief of anxiety, increased energy, strengthening of bones, improved heart health, lower blood pressure, proper blood sugar balance, improved stress response, enhanced wound healing, improved headaches/migraines, better, more restful sleep, improved PMS symptoms.

### Selenium

Selenium is an important nutrient for the thyroid gland is a crucial factor in the production of the body's main antioxidant, glutathione, and required for proper immune system function (through selenoproteins). As well, selenium is required to maintain proper peristalsis and to decrease inflammation throughout the body. Most crucial, though, may be the role selenium plays in the brain including memory, learning, cognition, and certain motor functions. Specifically, neuronal cells, utilizing γ-aminobutyric acid (GABA) as their primary means of signaling where GABA-utilizing neurons, reside in largest numbers in the brain regions: hippocampus, cerebral cortex and cerebellum and, likewise carry the ability to have the largest impact on neurological functions and brain functions in these areas. Too, selenium and selenoproteins have been noted to counter the over-potentiation of glutamate excitatory system where over-potentiation of glutamate-dependent neurons is a hallmark of Alzheimer's disease, epilepsy and other severe neurological disorders.

Symptoms of deficiency: low thyroid function, fatigue, brain fog, constipation, inflammation, poor immunity
Benefits: increased energy, improved brain function, proper thyroid function, healthy digestion, more robust immune function, anti-aging (e.g. improved cognition) to anti-inflammatory actions.

### Zinc

Zinc is required for the proper growth and maintenance of the human body. It is found in several systems and biological reactions and it is needed for proper immune function, wound healing, blood clotting, and thyroid function. Zinc, a transition metal, is a structural component of many proteins and is involved in the catalytic activity of cell enzymes. Zinc is an essential trace element the deficiency of which has been associated with major depressive disorder (MDD) at least partially explained by its function as an allosteric receptor channel modulator.¹⁹ Zinc is further a cofactor for the aromatase enzyme that converts estrogen to testosterone that can help women on birth control increase libido. Also, zinc deficiency has a definitive impact on androgen metabolism and aromatization, androgen and estrogen receptor binding and circulating levels of reproductive hormones where dihydrotesterone is increased but estradiol from testosterone is greater (where the majority of testosterone is taken up across the liver cell membrane and is converted by aromatase to estradiol) and adequate cofactor zinc produces the opposite (i.e. lower levels of circulating estradiol).

Symptoms of deficiency: sleep disruption, low libido, weight gain, inflammation, diarrhea, low insulin levels, irritability, hair loss, dry skin, growth retardation, infection, nausea and taste and smell disturbances
Benefits: correct thyroid functioning, boosts in immune system function, improved libido improved skin conditions, decreased inflammation, remuneration of neurodegenerative diseases, proper GI function, antioxidant activity and proper wound healing

### Calcium-D-Glucarate

Calcium-D-glucarate is similar to a naturally occurring chemical called glucaric acid and is made through a combination of glucaric acid and calcium. Calcium-D-glucarate is a substance that helps the liver detoxify hormones such as estrogen. When taking hormones, it is important to not only manage the proper absorption and metabolism of exogenous, as well as endogenous, hormones but also support the liver's ability to clear hormones out of the body via excretion.²³ Plainly higher levels of estrogen are introduced into the body, or increased levels are perceived by the body through improper elimination and secretion, these ‘high estrogens’, most closely associated with taking hormonal birth control, leads to dose-dependent and largely parallel increases in GI upset, headaches, weight gain and mood problems.

Symptoms of deficiency: poor estrogen detoxification, hormonal imbalances, skin, hair and nail abnormalities, abnormal heart rhythm, weight gain and break through bleeding
Benefits: detoxification of high hormones levels including excessive estrogen, improved liver function, healthy cholesterol levels, support of phase II detoxification and clearing of environmental toxins in the liver

### Phytochemical

### Broccoli Sprout Extract (Sulforaphane)

A phytochemical derived from the isothiocyanate group of organosulfur compounds, sulforaphane is found primarily in cruciferous (green leafy) vegetables (e.g. brassica vegetables) such as arugula, Brussel sprouts, kale, various greens (mustard, turnip and collard), cabbage and broccoli. Broccoli sprout extract, namely, is a concentrated source of sulforaphane as well as vitamin C, soluble fiber and other nutrients. Sulforaphane is one of the most powerful supports to the cell's powerhouse, the mitochondria, due to its ability to upregulate the body's anti-oxidant complex called the Nrf2/ARE complex and thus raise the body's main antioxidant and detoxifier-glutathione.^{10,11} Specifically, it has been noted that Nrf2, among other functions, regulates the transcription of glutathione (as well as GSH production, utilization and regeneration) and thioredoxin antioxidant systems as well as participation in phase I and phase II detoxification of endogenous and exogenous compounds, NADPH regeneration and heme metabolism (through iron sequestration) representing a critical regulator of cellular defense mechanisms against oxidative stress. Further, it has been noted that brassica vegetables have been implicated in the removal of various xenobiotics through the induction of cytochrome p450 (CYP) 1A2, N-acetyltransferase 2 (NAT2) and xanthine oxidase.^{26,28,29,30} As well, sulforaphanes work symbiotically with naturally occurring antioxidants (ex. Vit E and Co-Q) to potentiate each's antioxidant capacity within the body.²⁷

Symptoms of deficiency: inflammation, fatigue, mood disturbances, digestive distress, dermatitis, acne, weight gain, tender breast and breakthrough bleeding associated with poor estrogen metabolism.
Benefits: cardio protective effects associated with the predominant pre-menopausal circulating estrogen 17β-estradiol, detoxifies other environmental exposures²⁹, healthy cholesterol levels,³² supports the body's natural defenses against cell damage and oxidative stress²⁸, (quinone) detoxification²⁷, boosts antioxidants and anti-inflammatory compounds²⁸, improves liver and pancreatic β-Cell function^{28,31}, anti-cancer properties,²⁹ boosts brain function, reduces oxidative stress and super oxide dismutase and upregulation of the Nrf2/ARE complex (anti-oxidant complex).

### Alkaloid

piperine (Bioperine®)

Piperine, or mixtures containing piperine, have been utilized for centuries by ayurvedic practitioners for their ability to enhance the bioavailability, blood (plasma) levels and concentrations and overall efficacy of a number of pharmaceuticals, nutrients and nutritionally-based compounds by facilitating improved gastrointestinal absorption leading to insufficient “fuel” for proper metabolic functioning. Derived primarily from black pepper or extract of black pepper, piperine has the stated ability to improve the utilizable amount of certain nutrients in the body. This has been explained though one of two mechanisms (a) improved absorption (through increased rapidity of absorption, decreased destruction and facilitated active and passive transport and (b) downregulation of inhibitory enzymes thereby slowing or inhibition of inactivation or elimination (See U.S. Pat. Nos. 5,536,506, 5,744,161 and 5,972,382 issued to Majeed et. al). Specific to the present invention, is improved GI absorption of both fat soluble (e.g. Vitamin E) and water-soluble vitamins (e.g. B Vitamins and Vitamin C) where enhanced absorption is further augmented by increased retention and/or decreased excretion and overall received nutrient levels are at an increased utilizable level that are more available for requisite metabolic and hormonal consumption and incorporation into vital functions. Plainly, piperine can serve the dual role of fortifying diet derived nutrition as well as supporting and supplementing exogenously derived nutrients. Of particular importance, both selenium and B6 have shown marked elevations, respectfully, in the presence of piperine both having consequential beneficial effects as outlined above.

Symptoms of deficiency: decreased absorption or suboptimal absorption of nutrients and lower serum levels of nutrients
Benefits: increased absorption of nutrients, higher levels of nutrients in the bloodstream, microbiome balance

### PREFERRED EMBODIMENTS

In one preferred embodiment, the present nutraceutical invention is utilized to reintroduce deficient nutrients into those women taking hormonal contraception in order to remunerate deleterious effects of insufficient levels of B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin) and Vitamins C & E and minerals magnesium, selenium and zinc caused by hormone contraception.

In yet another embodiment, the present nutraceutical invention seeks to introduce deficient nutrients, B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin) and Vitamins C & E and minerals magnesium, selenium and zinc, into the women taking exogenous hormones (hormonal contraceptives or hormone replacement) as a means to (1) provide for a homeostatic state through the provision of sufficient nutrient supplementation (as would be seen in woman not taking hormonal contraception) and (2) as an adjunctive means to ensure proper functioning of hormonal pathways responsible for the absorption, metabolism and excretion of endogenous as well as exogenous hormonal substances, compounds and intermediaries.

It is another goal of the present invention to provide a nutraceutical supplement incorporating those nutrients known to be deficient in women taking hormonal birth control (i.e. B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin) and Vitamins C & E and minerals Magnesium, Selenium and Zinc for those woman taking hormone replacement.

It is another goal of inventor to augment the above formulation (i.e. B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin) and Vitamins C & E and minerals magnesium, selenium and zinc) with sulforaphane in efforts to aid in the body's upregulation of anti-oxidant complex (i.e. the Nrf2/ARE complex) and thus raise the body's primary antioxidant and detoxifier (glutathione), promote cardio protective effects associated with the predominant pre-menopausal circulating estrogen 17β-estradiol, detoxify environmental exposures , ensure and promote healthy cholesterol levels, support the body's natural defenses against cell damage and oxidative stress, generalized detoxification, facilitate antioxidants and anti-inflammatory compounds, improve liver and pancreatic β-Cell function in addition to potential anti-cancer properties, boosts to proper brain function and work symbiotically with other naturally occurring antioxidants.

It is another goal of inventor to augment the above formulation (i.e. B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin) and Vitamins C & E and minerals magnesium, selenium and zinc) with calcium-d-glucarate in efforts to aid support the liver's ability to clear hormones (e.g. estrogen and progestin) out of the body via excretion thereby lessening or avoiding the injurious effects of excess circulating hormones.

It is yet another goal of inventor to incorporate piperine (Bioperine®) into the above formulae (i.e. B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin) and Vitamins C & E and minerals magnesium, selenium and zinc with sulforaphane) in order to enhance the bioavailability of the above compounds to facilitate improved gastrointestinal absorption thus providing increased levels over non-facilitated (i.e. piperine absent) absorption. Also, piperine has the dual effect of downregulation of inhibitory enzymes thereby slowing or inhibiting substance inactivation or elimination thereby supporting circulating levels of the above, namely selenium and vitamin B6, heightening their positive effects (e.g. increased energy, improved brain function, proper thyroid function, healthy digestion, more robust immune function, in the case of selenium, and decreased nausea and vomiting, improved mood, more efficient processing of hormones, improved PMS symptoms, for B6).

It is another goal of the present formulation and method of use to reduce or obviate the adverse effects of oxidative stress on the body through the provision of exogenous antioxidants, the support of endogenous antioxidants (glutathione) and supportive cofactors assisting in the production and maintenance of antioxidant (e.g. selenium, B2 Vitamin C and Vitamin E) activity, either directly or indirectly (e.g. sulforaphanes), through the ongoing supplanting of necessary nutrients included within the present formulation.

It is another goal to appropriate the use of sulforaphanes to regulate the transcription of naturally occurring antioxidant GSH/glutathione (as well as GSH production, utilization and regeneration) and thioredoxin antioxidant systems with participation in phase I and phase II detoxification of endogenous and exogenous compounds, NADPH regeneration and heme metabolism (through iron sequestration) representing a critical regulator of cellular defense mechanisms against oxidative stress, removal various xenobiotics and to work symbiotically with naturally occurring antioxidants (ex. Vit E and Co-Q) to augment and potentiate their inherent antioxidant capacity.

It is yet another goal to utilize piperine (Bioperine®) to accentuate and reinforce the antioxidant activity and restorative function of the present formula, though increased bioavailability and downregulation of inhibitory enzymes slowing nutrient inhibition, inactivation or elimination, in order to stymie or reverse the undesirable effects of oxidative stress and/or to counter the potential increase in undesirable and potentially harmful consequences due to hormonal contraception implementation (e.g. K2, Vitamin A, copper and iron excess).

In one preferred embodiment, the present invention is comprised of the following formula:
B1 (Thiamin): 4.8 mg
B2 (R-5-P/riboflavin): 4 mg
B3 Niacin: 16 mg
B5 (Pantothenic Acid): 5 mg
B6 (Pyridoxine) as P-5-P: 10 mg
B7 (Biotin): 60 mcg
B9 Folic Acid-Methylfolate/methyltetrahydrafolate: 400 mcg
B12 (cyanocobalamin) as methylcobalamin: 10 mg
Vitamin C (L-ascorbate): 200 mg
Vitamin E (alpha-tocopherol and mixed tocopherols): 30 mg
Magnesium Malate: 50 mg
Selenium (selenomethionine): 55 mcg
Zinc bysglycinate: 15 mg
Calcium D Glucarate: 100 mg
Organic broccoli sprout powder(sulforaphane): 25 mg
Bioperine® (piperine): 5 mg

In yet another preferred embodiment, the present invention is comprised of the following formula including the below ranges:
B1 Thiamin: 0.01-50 mg
B2 (R-5-P/riboflavin): 0.01-500 mg
B3 Niacin: 0.01-1 gram
B5 (Pantothenic Acid): 500 mcg-20 mg
B6 as P-5-P: 0.01-100 mg
B7 Biotin: 5 mcg to 300 mcg
Methylfolate: 0.01-2 mg
B12 as methylcobalamin: 0.01-1 mg
Vitamin C (L-ascorbate): 0.01-4 grams
Vitamin E (alpha-tocopherol and mixed tocopherols): 0.01-100 mg
Magnesium Malate: 0.01-500 mg
Selenium (selenomethionine): 0.01-200 mcg
Zinc bysglycinate: 0.01-40 mg
Calcium D Glucarate: 0.01-1 gram
Organic broccoli sprout powder (sulforaphane): 0.01-300 mg
Bioperine® (piperine) 250 mcg-25 mg

### REFERENCES

### Background

1. Reading, B. Growth in World Contraceptive Use Stalling; 215 Million Women's Needs Still Unmet. Earth Policy Institute. 2012

2. Brynhildsen, J. Combined hormonal contraceptives: prescribing patterns, compliance, and benefits versus risks. Ther Adv Drug Saf. 2014 October; 5(5): 201-213.

3. Marc Dhont (2010) History of oral contraception, The European Journal of Contraception & Reproductive Health Care, 15:sup2, S12-S18, DOI: 10.3109/13625187.2010.513071

4. Griswold v. Connecticut, 381 US 479 (1965) stating that the Constitution does not explicitly protect a general right to privacy, the various guarantees within the Bill of Rights create penumbras, or zones, that establish a right to privacy. Together, the First, Third, Fourth, and Ninth Amendments, create a new constitutional right, the right to privacy in marital relations. The Connecticut statute conflicts with the exercise of this right and is therefore null and void.

5. Baird v. Eisenstadt, 405 US 438 (1972) “If the right of privacy means anything”, wrote Justice William J. Brennan, Jr. for the majority, “it is the right of the individual, married or single, to be free from unwarranted governmental intrusion into matters so fundamentally affecting a person as the decision whether to bear or beget a child.”

6. Brunton L L, Bjorn Knollmann, et al.: Goodman and Gilman's The Pharmacological Basis of Therapeutics. 13^{th} Edition. 2017

7. Kimberly Daniels, William Mosher, Jo Jones. Contraceptive Methods Women Have Ever Used: United States, 1982-2010. National Health Statistics Reports. Number 62. Feb. 14, 2013.

8. Kimberly Daniels, Jill Daugherty, and Jo Jones. Current Contraceptive Status Among Women Aged 15-44: United States, 2011-2013. NCHS Data Brief. No. 173. December 2014.

9. Mohn, E, Kern, H, Saltzman, et al. Evidence of Drug-Nutrient Interactions with Chronic Use of Commonly Prescribed Medications: An Update. Pharmaceutics 2018 Mar. 20; 10(1).

10. Palmery, M, Saraceno A, Vaiarelli, A and Carlomagno, G. Oral Contraceptives and changes in Nutritional Requirements. Eur Rev Med Pharmacol Sci. 2013 July; 17(13):1804-13.

11. Fahey et al. Broccoli sprouts: An exceptionally rich source of inducers of enzymes that protect against chemical carcinogens. Proc Natl Acad Sci USA. 1997 Sep. 16; 94(19): 10367-10372.

12. Carrasco-Pozo et al. Sulforaphane is anticonvulsant and improves mitochondrial function. J Neurochem. 2015 December; 135(5):932-42. doi: 10.1111/jnc.13361. Epub 2015 Oct. 8.

13. Tarozzi et al. Sulforaphane as a Potential Protective Phytochemical against Neurodegenerative Diseases. Oxidative Medicine and Cellular Longevity Vol. 2013, Article ID 415078, 10 pages.

14. Hershkowitz E, Markel A. Thiamine—“The Road Experience” of the vitamin as a manifestation of deficiency in a world of abundance. Harefuah. 2015 October; 154(10):661-4, 674.

15. Wisniewski J et al. In-depth quantitative analysis and comparison of the human hepatocyte and hepatoma cell line HepG2 proteomes. Journal of Proteomics

16. Wang et al. High thiamine diphosphate level as a protective factor for Alzheimer's disease. Neurol Res. 2018 May 2:1-8

17. Marcus, R. and Coulston, A. (1996) Water Soluble Vitamins—The Vitamin B Complex and Ascorbic acid. J. Hardman, L. Limbird, P. Molinoff, R. Rudden, A. Gilman (Eds.), Goodman and Gilmans-The Pharmacological Basis of Therapeutics. (p. 1555). New York. McGraw Hill Companies.

18. Rucker R B, Steinberg F M, Johnston C S. Handbook of Vitamins. Fourth. CRC Press; 2007. Ascorbic acid.

19. Anderson, et al. Effects of oral contraceptives on vitamin metabolism. Adv Clin Chem. 1976; 18:247-87.

20. Islam M R. Alterations of serum macro-minerals and trace elements are associated with major depressive disorder: a case-control study. BMC Psychiatry. 2018 Apr. 10; 18(1):94

21. Egoramaiphol S, Migasena P, Supawan V. Effect of oral contraceptive agents on thiamine status. J Med Assoc Thai. 1985 January; 68(1):19-23.

22. Eyad, et al. Riboflavin Has Neuroprotective Potential: Focus on Parkinson's Disease and Migraine. Front Neurol. 2017; 8:333.

23. Calcium-D-glucarate. Altern Med Rev. 2002 August; 7(4):336-9.

24. Wade, A E, Evans, J S. The influence of thiamin deficiency on the metabolism of the oral contraceptive mestranol [3-methoxy-17-ethynyl-1,3,5(10)-estratrien-17 beta-ol] by female rat liver enzymes. Steroids. 1977 August; 30(2):275-83.

25. Condo et al. Riboflavin prophylaxis in pediatric and adolescent migraine. J Headache Pain. 2009 October; 10(5):361-5. doi: 10.1007/s10194-009-0142-2. Epub 2009 Aug. 1.

26. Lampe, J. et al. Brassica vegetables increase and apiaceous vegetables decrease cytochrome P450 1A2 activity in humans: changes in caffeine metabolite ratios in response to controlled vegetable diets. Carcinogenesis, Volume 21, Issue 6, 1 Jun. 2000, Pages 1157-1162.

27. Carrasco-Pozo, C. et al. Sulforaphane Protects against High Cholesterol-Induced Mitochondrial Bioenergetics Impairments, Inflammation, and Oxidative Stress and Preserves Pancreatic β-Cells Function. Oxid Med Cell Longev. 2017; 2017:3839756. doi: 10.1155/2017/3839756. Epub 2017 Mar. 12.

28. Abdull Rzzais A F, et al. Isothiocyanates and Xenobiotic Detoxification. Mol Nutr Food Res. 2017 Dec. 30.

29. Zhou R, Lin J and Wu D. Sulforaphane induces Nrf2 and protects against CYP2E1-dependent binge alcohol-induced liver steatosis. Biochim Biophys Acta. 2014 January; 1840(1):209-18.

30. Tian et al. Sulforaphane Improves Abnormal Lipid Metabolism via Both ERS-Dependent XBP1/ACC &SCD1 and ERS-Independent SREBP/FAS Pathways. Send to Mol Nutr. Food Res. 2018 March; 62(6):e1700737.

I claim:

1. A formulation for nutrient supplementation for women taking exogenous hormones consisting of:
B1 Thiamin: 0.01-50 mg
B2 (R-5-P/riboflavin): 0.01-500 mg
B3 Niacin: 0.01-1 gram
B5 (Pantothenic Acid): 500 mcg-20 mg
B6 as P-5-P: 0.01-100 mg
B7 Biotin: 5 mcg to 300 mcg
Methylfolate: 0.01-2 mg
B12 as methylcobalamin: 0.01-1 mg
Vitamin C (L-ascorbate): 0.01-4 grams
Vitamin E (alpha-tocopherol and mixed tocopherols): 0.01-100 mg
Magnesium Malate: 0.01-500 mg
Selenium (selenomethionine): 0.01-200 mcg
Zinc bysglycinate: 0.01-40 mg
Calcium D Glucarate: 0.01-1 gram
Organic broccoli sprout powder (sulforaphane): 0.01-300 mg
Bioperine® (piperine) 250 mcg -25 mg

2. The formulation of claim 1 wherein the ingredients are in a pure or synthetic form.

3. The formulation of claim 1 wherein said formulation further comprises:
a pharmaceutically acceptable carrier or carriers, a pharmaceutically acceptable excipient or excipients, a pharmaceutically acceptable filler or fillers, a pharmaceutically acceptable flavoring agent or flavoring agents, a pharmaceutically acceptable coloring agent or coloring agents, a pharmaceutically acceptable stabilizing agent or stabilizing agents, a pharmaceutically acceptable binder or binders, a pharmaceutically acceptable disintegrant or disintegrants or a combination thereof.

4. The formulation of claim 3, wherein said formulation is administered orally, intramuscularly, intradermally, intravenously, nasally, subcutaneously or intraperitoneally before, concurrently with or subsequent to introduction of exogenous hormones.

5. The formulation of claim 1 wherein said formulation consists of:
B1 (Thiamin): 4.8 mg
B2 (R-5-P/riboflavin): 4 mg
B3 (Niacin): 16 mg
B5 (Pantothenic Acid): 5 mg
B6 (pyridoxal-5-phosphate) in the form of P-5-P: 10 mg
B7 (Biotin): 60 mcg
B9 (Folic Acid) in the form of Methylfolate or methyltetrahydrafolate: 400 mcg
B12 (cyanocobalamin) as methylcobalamin: 10 mg
Vitamin C as L-ascorbate: 200 mg
Vitamin E as alpha-tocopherol or mixed tocopherols): 30 mg
Magnesium as magnesium malate: 50 mg
Selenium as selenomethionine: 55 mcg
Zinc as zinc bysglycinate: 15 mg
Calcium as Calcium D Glucarate: 100 mg
Organic broccoli sprout powder (sulforaphane): 25 mg
Bioperine® (piperine): 5 mg

6. The formulation of claim 5 wherein the ingredients are in a pure or synthetic form.

7. The formulation of claim 5 wherein said formulation further comprises:
a pharmaceutically acceptable carrier or carriers, a pharmaceutically acceptable excipient or excipients, a pharmaceutically acceptable filler or fillers, a pharmaceutically acceptable flavoring agent or flavoring agents, a pharmaceutically acceptable coloring agent or coloring agents, a pharmaceutically acceptable stabilizing agent or stabilizing agents, a pharmaceutically acceptable binder or binders, a pharmaceutically acceptable disintegrant or disintegrants or a combination thereof.

8. The formulation of claim 5, wherein said formulation is administered orally, intramuscularly, intradermally, intravenously, nasally, subcutaneously or intraperitoneally, before, concurrently with or subsequently to introduction of exogenous hormones.

9. A method for supplying to a woman a supplement and nutraceutical compound, consisting of a pharmaceutically acceptable amount of B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin), Vitamins C & E, magnesium, selenium, zinc, sulforaphane, piperine and calcium-d-glucarate, to reintroduce and maintain deficient nutrients in those women taking exogenous hormones including the steps of:
collecting a first specimen from said woman to determine the nutrient status of a patient before, after or during exogenous hormone ingestion;
supplying said supplement and nutraceutical compound consisting of B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin), Vitamins C & E, magnesium, selenium, zinc, sulforaphane, piperine and calcium-d-glucarate to said woman;
collecting a second specimen from said woman to determine nutrient status some time after the ingestion of both exogenous hormones and said nutraceutical compound;
comparing said first specimen from said second specimen to determine improvement or decline in nutritional status; and
adjusting pharmaceutically acceptable amounts of said supplements and nutraceutical compounds to match the surplus and deficiencies expressed in each patient.

10. The method of claim 9 wherein said supplement and nutraceutical compound is administered to a woman taking exogenous hormones in order to remunerate deleterious effects of insufficient levels of B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin), Vitamins C & E and magnesium, selenium and zinc by facilitating proper mitochondrial function and support, providing requisite nutrient transport, absorption, metabolism and excretion while working as a ubiquitous cellular antioxidant and hormonal detoxification mediator all while assisting in maintaining proper microbiome and biochemical balance within the body.

11. The method of claim 9 wherein said nutraceutical compound is administered to a woman taking exogenous hormones in order to remunerate deleterious effects of insufficient levels of B1 (Thiamine), B2 (Riboflavin), B3 (Niacin), B5 (Pantothenic acid) B6 (Pyridoxine), B7 (Biotin), B9 (Folic Acid in the form of Methylfolate), B12 (Methylcobalamin), Vitamins C & E and magnesium, selenium and zinc to remunerate the effects of the depletion of essential nutrients and the resulting biochemical imbalances that produce dysfunctional metabolic processes within the female body adversely affecting mood, skin, hair, energy level, libido, gastrointestinal function related directly or indirectly to hormone production, metabolism and regulation and excretion and/or lack thereof.

12. The method of claim 9 wherein said nutraceutical compound is administered to a woman taking exogenous hormones to halt or reverse the deleterious effects of oxidative stress and support antioxidant activity and glutathione production and maintenance to lessen or reverse symptoms of excess exogenous and indigenous hormones including, but not limited to, headaches, fatigue, decreased neurotransmitter activity, weight gain gastrointestinal dysfunction, decreased liver function and resultant hormone metabolism insufficiencies, decreased libido and cardiovascular health issues.

13. The method of claim 9 wherein said nutraceutical compound is administered to a woman taking exogenous hormones to halt or reverse the deleterious effects of increased levels of K2 (menaquinone), Vitamin A, Iron and Copper and to support the proper ratio of calcium to magnesium in the body.

14. The method of claim 9 wherein sulforaphane is utilized to support the mitochondria, upregulate the Nrf2/ARE complex and increase levels of glutathione through increase in glutathione transcription, production, utilization and regeneration, supporting the thioredoxin antioxidant systems, NADPH regeneration and heme metabolism and symbiotic reinforcement of naturally occurring and exogenously suppled antioxidants.

15. The method of claim 9 wherein piperine is utilized to enhance the bioavailability of the individual constituents of said nutraceutical compound through (a) improved constituent absorption and (2) downregulation of inhibitory enzymes thereby slowing or inhibition of inactivation or elimination of constituents, singly and wholly.

16. The method of claim 9 wherein calcium-d-glucarate is utilized to support the liver's ability to clear exogenous and endogenous hormones out of the body via excretion thereby lessening or avoiding the injurious effects of excess circulating hormones.

17. The method of claim 9 wherein said supplement and nutraceutical compound is suppled to a woman taking exogenous hormones (hormonal contraceptives or hormone replacement) as a means to (1) provide for a homeostatic state through the provision of sufficient nutrient supplementation (as would be seen in woman not taking hormonal contraception) and (2) as an adjunctive means to ensure proper functioning of hormonal pathways responsible for the absorption, metabolism and excretion of endogenous as well as exogenous hormonal substances and intermediaries.

18. The method of claim 9 wherein said supplement and nutraceutical compound is suppled to a woman taking exogenous hormones as a means to ameliorate and/or obviate the intractable effects of exogenous estrogens and progestins on the body by facilitating proper mitochondrial function and support, providing requisite nutrient transport, absorption, metabolism and excretion while working as a cellular antioxidant and hormonal detoxification mediator all while assisting in maintaining proper microbiome and biochemical balance within the body.

19. The method of claim 9 wherein supplying to a woman a supplement and nutraceutical compound, consisting of a pharmaceutically acceptable amount of B1 (Thiamine), B2 (Riboflavin), B3 (Niacin), B5 (Pantothenic acid), B6 (Pyridoxine), B7 (Biotin), B9 (Folic Acid in the form of Methylfolate), B12 (Methylcobalamin), Vitamins C & E, magnesium, selenium, zinc, sulforaphane, piperine and calcium-d-glucarate serves the following primary functions:
B1 (Thiamine): used to generate energy via the Krebs cycle, mitochondrial pathways and the Pentose Phosphate Pathway, decease nausea creating lactate, support proper nervous system and neurotransmitter function and control exogenous hormone metabolism rates;
B2 (Riboflavin): neuroprotective function, cofactor in electron carriers and helps to regulate redox reactions and other metabolic pathways, decreases oxidative stress, facilitates vitamin B3 (niacin), B6, B9 (folate) and iron and tryptophan status, enhances mitochondrial function, participates in the production of glutathione to regulate inflammatory control, immune balance and detoxification of endogenous and exogenous toxins via phase 1 and 2 detoxification in the liver;
B3 (Niacin): supports mitochondrial function and genomic stability, works interdependently with riboflavin, and is essential for neuronal health;
B5 (Pantothenic acid): essential to formulate CoA for fat, carbohydrate and protein synthesis, metabolism and hormone generation, synthesis of Co-enzyme A (CoA) and is vital to energy derivation for pyruvate entering the TCA cycle (tricarboxylic acid cycle) also titled the citric acid or Krebs cycle;
B6 (Pyridoxine): protein metabolism, nervous system health (e.g. neurotransmitter synthesis), hemoglobin synthesis and function (transport of oxygen), glucose metabolism, lipid metabolism, DNA production and gene expression, hormone regulation, histamine synthesis, mood-regulation, immune balancing and inflammatory controlling amino-acid tryptophan metabolism (with the continued facilitation of B6 with Vitamin C, zinc and magnesium) and homocysteine balance;
B7 (Biotin): fats, carbohydrates and amino acids metabolism;
B9 (Folic Acid in the form of Methylfolate): maintenance of healthy DNA, RNA and amino acids and is responsible for appropriate energy production (ATP) and red blood cell formation along with regulation;
B12 (Methylcobalamin): methylation cycle for healthy DNA production, metabolism of every cell in the body, coating nerves in myelin sheath, production of red blood cells in the bone marrow;
Vitamin C: antioxidant in the body used to fight free radical damage and to boost levels of the body's main antioxidant, glutathione, crucial nutrient for the production of carnitine which modulates inflammation in the mitochondria, produces the neurotransmitters, dopamine, norepinephrine and epinephrine, and is important for collagen production for tendons, ligaments and skin integrity Vitamin E: antioxidant that protecting cells, tissues and organs from free radical damage along with aiding in recycling glutathione, protects skin, lungs and cell membranes, enhances immune function and improves libido;
magnesium: antioxidant, maintains muscle and nerve function, potentiate energy production, prevent muscle cramps, supports heart rhythm, lowers oxidative stress, strengthens bones, regulates blood sugar levels and controls calcium levels;
selenium: maintains thyroid gland function, immune function GI function, brain and neurologic function,
zinc: immune function, mental health, decreases circulating estrogen through inhibition of aromatase;
sulforaphane: upregulate the body's anti-oxidant Nrf2/ARE complex, regulates the transcription of glutathione (as well as GSH production, utilization and regeneration) and thioredoxin antioxidant systems as well as participation in phase I and phase II detoxification of endogenous and exogenous compounds, and work symbiotically with naturally occurring antioxidants;
piperine: improved absorption (through increased rapidity of absorption, decreased destruction and facilitated active and passive transport and downregulation of inhibitory enzymes thereby slowing or inhibition of inactivation or elimination; and
calcium-d-glucarate: helps the liver detoxify endogenous and exogenous hormones, decreasing side effects associated with excess hormones.
